(19) United States
(12) Patent Application Publication
(10) Pub. No.: US 2014/0275842 A1
(43) Pub. Date: Sep. 18, 2014

(54) Connected Surface with Sensors

(71) Applicant: Beam Technologies, LLC, Louisville, KY (US)

(72) Inventors: Alexander D. Curry, Louisville, KY (US); Alex X. Frommeyer, Louisville, KY (US); Daniel E. Dykes, Louisville, KY (US)

(73) Assignee: Beam Technologies, LLC, Louisville, KY (US)

(21) Appl. No.: 14/210,245

(22) Filed: Mar. 13, 2014

Related U.S. Application Data
(60) Provisional application No. 61/778,512, filed on Mar. 13, 2013.

(51) Int. Cl. A61B 5/00; A61B 5/021; A61B 5/01

(52) U.S. Cl. CPC A61B 5/6892; A61B 5/01; A61B 5/02141; A61B 5/445; A61B 5/4509; A61B 5/4872
USPC 600/301

(57) ABSTRACT

A connected surface is provided with various sensors for measuring multiple health statistics including heart rate, blood oxygen saturation, respiration rate, blood pressure, bone density, weight, balance, percent body fat, skin ulcers, stress level, pressure points, electrical heart activity, thermal imaging, and any combination thereof. Further, the connected surface may be comprised in flooring, a bath mat, a weight scale, a dedicated system, or any combination thereof. The connected surface also provides a user identification system utilizing capacitive coupling of the human body and voice feedback based on the user identified. The connected surface is comprised in a system including a data transfer medium (i.e. “smartphone”) and the Cloud, which allows for data transfer between multiple platforms from the connected surface.

### CROSS-REFERENCE TO RELATED APPLICATIONS

This application claims priority to U.S. Provisional Application No. 61/778,512 filed on Mar. 13, 2013.

### BACKGROUND OF THE INVENTION

This invention relates to measuring health statistics, particularly relating to a connected surface with sensors. In particular, the invention relates to the measurement of health statistics when the user is standing on the connected surface.

A person spends a significant amount of time each day just preparing for the day. Currently, this time provides little advantage beyond hygiene and cosmetic purposes. Often, a person spends much of this time positioned standing in front of a bathroom sink or other item such that an opportunity to measure health statistics is provided since the user is standing still for an amount of time. Current methods for collecting health statistics as part of the daily routine are largely nonexistent or require the user to deliberately step on a scale to obtain weight and possible body fat percentage. Current methods are limiting in both the health statistics that are measured and the passive nature of data collection.

### BRIEF SUMMARY OF THE INVENTION

The invention aims to provide a connected surface with sensors. The connected surface allows a user to stand on the surface to collect data related to health statistics. The user stands on the surface and the various sensors act to measure health statistics about the user. The sensors communicate with the data processing unit, which transmits the data using the transmitter. The data is transmitted to the cloud and/or a data transfer medium. Additionally, the connected surface is comprised in a capacitive coupling user identification system to easily identify the user to the connected surface allowing for use by multiple users.

The health statistics that can be measured are heart rate, blood oxygen saturation, respiration rate, blood pressure, bone density, weight, balance, percent body fat, skin ulcers, stress level, pressure points, electrical heart activity, thermal imaging, and any combination thereof.

Accordingly several advantages are to provide a connected surface, to provide sensors to measure various health statistics, to provide data communication with the cloud and data transfer mediums, and to provide user identification to allow for multiple users. Still further advantages will become apparent from a study of the following descriptions and the accompanying drawings.

### BRIEF DESCRIPTION OF DRAWINGS

FIG. 1 is a perspective view of a connected surface showing surface the user stands on as described in multiple embodiments and alternatives.

FIG. 2 is a schematic view of the components of a connected surface with an oximetry sensor as described in multiple embodiments and alternatives.

FIG. 3 is a schematic view of the components of a connected surface with an ultrasonic sensor as described in multiple embodiments and alternatives.

FIG. 4 is a schematic view of the components of a connected surface with a weight sensor as described in multiple embodiments and alternatives.

FIG. 5 is a schematic view of the components of a connected surface with a thermal imaging sensor as described in multiple embodiments and alternatives.

FIG. 6 is a schematic view of the components of a connected surface with a galvanic skin response sensor as described in multiple embodiments and alternatives.

FIG. 7 is a schematic view of the components of a connected surface with a body fat sensor as described in multiple embodiments and alternatives.

FIG. 8 is a schematic view of the components of a connected surface with an electrocardiogram sensor as described in multiple embodiments and alternatives.

FIG. 9 is a schematic view of the components of a connected surface with a pressure point sensor as described in multiple embodiments and alternatives.

FIG. 10 is a schematic view of the components of a connected surface with a temperature sensor as described in multiple embodiments and alternatives.

FIG. 11 is a schematic view of a connected surface system with user identification as described in multiple embodiments and alternatives.

FIG. 12 is a schematic view of a connected surface system with the cloud and a data transfer medium as described in multiple embodiments and alternatives.

### DETAILED DESCRIPTION OF THE INVENTION

The connected surface with sensors is encompassed in a plurality of embodiments that shall be discussed in the present section.

Referring to FIG. 1, A plurality of embodiments comprises a surface 103 that allows a user to stand on said surface 103 during normal daily activities, such as tooth brushing, drying after bathing, etc. Said surface 103 may be chosen from the group flooring, bath mat, weight scale, rug, dedicated system, and any combination thereof. Flooring is the walking surface of a room and may consist of various materials including wood, carpet, etc. A bath mat is a mat used on the floor of a bathroom that provides a warm, non-slip surface and may absorb certain amounts of water. A weight scale is a measuring device for determining the weight or mass of a person. A rug is a textile floor covering consisting of an upper layer attached to a backing. A dedicated system is a dedicated surface designed for the specific purpose of collecting data through various sensors and transmitting said data.

Additionally, the connected surface of the present invention comprises a data processing unit 216 having at least one collector, a storage medium, and at least one processor, wherein the collector, storage medium, and processor, respectively, collect, store, and process data. Accordingly, the data processing unit 216 is chosen from the group microprocessor, microcontroller, field programmable gate array (FPGA), digital signal processing unit (DSP), application specific integrated circuit (ASIC), programmable logic, and combinations thereof.

Additionally, in some embodiments, the collector of the data processing unit 216 is an electrically conductive wire, wherein the electrically conductive wire receives the electrical output of various sensors.

Moreover, the storage medium of the data processing unit 216 is comprised of volatile memory and non-volatile memory, wherein volatile memory is used for short-term storage and processing, and non-volatile memory is used for long-term storage. Accordingly, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

The processor of the data processing unit 216 is chosen from the group microprocessor and micro controller.

Furthermore, the connected surface comprises at least one data transmitter 224, such that the data can be transmitted to be used by another medium and data can be received from another medium. The data is packaged as at least one signal and transmitted to another medium. The data transmitter 224 is chosen form the group universal serial bus (USB), serial port, wired Ethernet port, radio frequency, microwave communication, infrared short-range communication, near field communication, and Bluetooth®.

Referring now to FIG. 2, the connected surface further comprises an oximetry sensor 237. Optionally, the oximetry sensor 237 is a reflective pulse oximeter or a transmissive pulse oximeter, wherein both types of oximetry sensors detect health statistics chosen from the group blood oxygen saturation, heart rate, respiration rate, blood pressure, blood flow, and any combination thereof.

The transmissive pulse oximeter comprises two distinct sides that are parallel with a space separating the two sides creating a measuring site such that a portion of the human body may be inserted between the two sides. The portion of the human body most often inserted in the measuring site is chosen from the group index finger, middle finger, ring finger, pinky finger, thumb, toe, ear lobe, and nose. Two light-emitting diodes (LED) are at least partially contained on the first parallel side creating an emitter. In some embodiments, the two LEDs produce beams of light at different frequencies, which include the range of about 600-750 nanometers (nm) and the range of about 850-1000 nm such that the frequencies produce red and infrared light, respectively. Additionally, the second parallel side comprises a photo detector positioned to be opposite of the emitter such that the photo detector receives the emitted light that passes through the measuring site. The photo detector determines the amount of red and infrared light received, thus determining the amount of red and infrared light absorbed. Accordingly, the amounts of red and infrared light are transmitted to the data processing unit 216 of the connected surface.

Optionally, the data processing unit 216 of the connected surface calculates the ratio of red light to infrared light after the emitted light passes through the measuring site and is received by the photo detector. The calculated ratio is compared to a data bank that relates the calculated ratio to blood oxygen saturation values. The heart rate is further determined by the amount of light absorption of the volume of arterial blood. As the heart pumps blood, the volume of arterial blood increases thus creating a pulsatile change in light absorption. The heart rate is determined by the frequency of pulsatile changes representing heart beats.

Optionally, the reflective pulse oximeter comprises one distinct side, referred to as the contact surface that comprises both the light emitter and the photo detector such that the emitted light travels into the measuring site and is reflected back to the photo detector. The reflective pulse oximeter allows the user to contact only one surface on the connected surface. Accordingly, the reflective pulse oximeter may be contacted by the user during the normal use of the connected surface.

Accordingly, the reflective pulse oximeter transmits the amounts of red and infrared light received by the photo detector via the transmitter to the data processing unit. Similarly, the ratio of red light to infrared light is calculated and compared to a data bank to correlate the ratio to a blood oxygen saturation value. Additionally, the heart rate of the user is determined in the same manner as described for the transmissive pulse oximeter.

In some embodiments of the reflective pulse oximeter, the contact surface is positioned to be flush with the portions of the connected surface surrounding the reflective pulse oximeter such that the handle and the reflective pulse oximeter are comprised in a smooth surface. Optionally, the contact surface is positioned to be raised above the portions of the connected surface surrounding the reflective pulse oximeter such that the reflective pulse oximeter is noticeably distinct from the portions of the connected surface surrounding it. Optionally still, the contact surface is positioned to be flush with the portions of the connected surface surrounding the reflective pulse oximeter, and at least a portion of the connected surface not directly surrounding the reflective pulse oximeter is raised such that the reflective pulse oximeter is located in at least a partial depression indicating where the user shall place his/her body part for contact with the contact surface.

In some embodiments, the oximetry sensor 237 may be a plurality of transmissive pulse oximeters. In some embodiments, the oximetry sensor may be a plurality of reflective pulse oximeters. Also, in some embodiments, the oximetry sensor may be a combination of at least one transmissive pulse oximeter and at least one reflective pulse oximeter.

As shown in FIG. 3, the connected surface comprises an ultrasonic sensor 341 that measures the bone density of the user from the heel of the foot. The ultrasonic sensor 341 includes at least one ultrasonic transducer and at least one ultrasonic detector. The ultrasonic transducer converts energy into ultrasound and emits said ultrasound, which is sound waves above the normal audible range of human hearing, typically with a frequency of 20 MHz or greater. Optionally, the ultrasonic transducer is a piezoelectric transducer that converts electrical energy into ultrasound by applying an alternating current (AC) across piezoelectric material, which holds the property of changing size when a voltage is applied to it. The application of alternating current to piezoelectric material provides a high frequency oscillation of the piezoelectric material. Consequently, very high frequency sound waves are produced by the high frequency oscillation of the piezoelectric material.

Additionally, the ultrasonic detector is a piezoelectric detector that receives ultrasound causing the piezoelectric material to oscillate at a high frequency, thus producing an electrical voltage indicative of the frequency of the ultrasound. Optionally, the piezoelectric transducer and the piezoelectric detector utilize the same body of piezoelectric material. Accordingly, the combined embodiment of the piezoelectric transducer and the piezoelectric detector is a piezoelectric transceiver, which performs the functions of both the piezoelectric transducer and the piezoelectric detector comprised in one singular body of piezoelectric material. Conversely, the piezoelectric transducer and the piezoelectric detector utilize separate bodies of piezoelectric material.

The piezoelectric material is chosen from the group Quartz, Berlinite (AlPO₄), Potassium sodium tartrate, Topaz (Al₂SiO₄(F, OH)₂), Gallium orthophosphate (GaPO₄), Langasite (La₃Ga₅SiO₁₄), Barium titanate (BaTiO₃), Lead titanate (PbTiO₃), Lead zirconate titanate (Pb[ZrₓTi₁₋ₓ]O₃, 0≦x≦1), Potassium niobate (KNbO₃), Lithium niobate (LiNbO₃), Lithium tantalite (LiTaO₃), Sodium tungstate (Na₂WO₃), Sodium potassium niobate (NaKNb), Bismuth ferrite (BiFeO₃), Sodium niobate (NaNbO₃), and Polyvinylidene fluoride (PVDF).

Optionally, the ultrasonic transducer is a magnetostrictive transducer comprising a magnetostrictive material, magnetizing coil, and magnetic enclosure, wherein the combination of the three elements completes a magnetic circuit. Magnetostrictive transducers utilize the magnetostrictive property of the magnetostrictive material to convert the magnetic energy of a magnetic field to ultrasound, which is sound waves above the normal audible range of human hearing, typically with a frequency of 20 MHz or greater. The magnetostrictive property is a material property, common to ferromagnetic materials, where the material is divided into uniform magnetic polarization domains, such that when a magnetic field is applied said domains shift and rotate causing the magnetostrictive material to change size at a high frequency, thus generating high frequency sound waves or ultrasound. In a magnetostrictive transducer, the magnetic field is provided by the magnetizing coil wrapped around the magnetostrictive material. The magnetic field of the magnetizing coil is produced by the input of electrical energy into the coil.

In some embodiments, the ultrasonic detector is a magnetostrictive detector comprising a magnetostrictive material, magnetizing coil, and magnetic enclosure, wherein the combination of the three elements completes a magnetic circuit. In the same manner as the magnetostrictive transducer, the magnetostrictive detector utilizes the magnetostrictive property of the magnetostrictive material to convert ultrasound to magnetic energy, which alters the magnetic field of the magnetizing coil, thus altering the electrical energy output of the magnetostrictive detector.

The magnetostrictive material is chosen from the group Cobalt, Terfenol-D, and Metglas 2605SC. In some embodiments, the magnetizing coil is manufactured from an electrically conductive material. Additionally, in some embodiments, the magnetostrictive transducer and the magnetostrictive detector utilize the same magnetostrictive material, magnetizing coil, and magnetic enclosure, consequently embodied as a magnetostrictive transceiver. Optionally, the magnetostrictive transducer and the magnetostrictive detector have separate magnetostrictive materials, magnetizing coils, and magnetic enclosures.

Optionally, the ultrasonic transducer is a capacitive actuator comprising two conductive plates on either side of a dielectric material, wherein electrical energy is passed from one conductive plate through the dielectric material to the second conductive plate. The passing of electrical energy across the conductive plates causes the conductive plates to acquire opposite charges, which further causes an attractive force to exist between the conductive plates. Electrical energy in the form of alternating current provides high frequency oscillation of the capacitive actuator, thus converting electrical energy into ultrasound.

In some embodiments, the ultrasonic detector is a capacitive actuator having the same properties as stated above. The process is reversed in the instance of the ultrasonic detector, such that ultrasound is received that affects the oscillation of the capacitive actuator, and the electrical energy passed between the two conductive plates through the dielectric material is altered as a result.

In some embodiments, the ultrasonic sensor is a microelectromechanical system (MEMS). A microelectromechanical system is characterized as a system comprising miniaturized mechanical and electro-mechanical elements that are fabricated using the techniques of microfabrication. A microelectromechanical system is further characterized as comprising miniaturized structures, referred to as microstructures; miniaturized sensors, referred to as microsensors; miniaturized actuators, referred to as microactuators; and microelectronics. Microsensors and microactuators are commonly referred to as microtransducers, which are miniaturized devices that convert energy from one medium to another, such as mechanical to electrical.

Accordingly, the ultrasonic transducer transmits ultrasound through the heel of the user into the user's bones and the ultrasound is reflected. The reflected ultrasound is received by the ultrasonic detector. The time between transmission and detection of ultrasound, essentially the speed of sound, is correlated to reference bone density data stored by the data processing unit.

Referring to FIG. 4, the connected surface comprises a weight sensor 450 to collect weight and balance measurements of the user. The weight sensor 450 is chosen from the group at least one strain gage, electronic analytical scale, capacitive scale, and any combination thereof. Accordingly, the weight sensor 450 is distributed across the connected surface such that weight measurements are made when the user is standing on the connected surface at any orientation. Further, the distributed weight sensor provides for balance measurement based on the differing pressure applied by the user's individual feet such that more pressure from one foot may indicate poor balance.

A strain gage is comprised of an insulating flexible backing with a metallic foil pattern affixed and is attached to the connected surface in a proper place with an adhesive, such as cyanoacrylate. As a load is applied to the connected surface, the connected surface deforms causing the foil of the strain gage to deform that results in an electrical resistance change, which is typically measured using a Wheatstone bridge. The resistance change is converted into a strain value using a gage factor and the strain value is used to calculate the load on the connected surface, which provides the weight of the user providing the load.

An electronic analytical scale measures the load on the connected surface by countering the load applied using an electromagnet to generate a force. The measurement of the counter force applied by the electromagnet and the electromagnet itself are often comprised in an electromagnetic force restoration sensor.

A capacitive scale comprises a capacitive sensor that is constructed from two parallel conductive plates separated by an insulator such that, in the active portion of the sensor, the insulator allows for an air gap between the parallel plates. Forces acting perpendicular to the plane of the parallel plates in the active region deform one or both conductors. Accordingly, the parallel plates move closer together or farther apart due to deformation, thus, changing the capacitance of the sensor. The change in capacitance is scaled by a reference factor that provides the weight of the item causing the capacitance change.

Referring now to FIG. 5, the connected surface further comprises at least one thermal imaging sensor 565, wherein the thermal imaging sensor 565 is located on the underside of the connected surface such that its field of view comprises the user standing on the connected surface. The thermal imaging sensor 565 detects radiation in the infrared of the electromagnetic spectrum, which is roughly 9,000-14,000 nanometers. The amount of radiation emitted increases with temperature, thus providing a differentiation between hot and cold areas. Accordingly, the thermal imaging sensor 565 allows the data processing unit 216 to identify hot spots on the user's body that correlate to potential ulcers. Often, foot ulcers are a symptom of diabetes and indicate the onset of more serious medical ailments.

Now referring to FIG. 6, the connected surface further comprises a galvanic skin response sensor 678 that measures the moisture level of the user's skin via the electrical conductance of the skin. The galvanic skin response sensor 678 measures the electrical conductance between two points by transmitting a small amount of current through the body that follows two paths, which are the surface of the skin and through the body. The conductance varies based on the response of the skin and muscle tissue to the stimuli. The conductance resulting from the test is correlated to reference values that can be personalized to individual users. The correlation to reference values provides indicators of stress and anxiety levels the user is experiencing.

As shown in FIG. 7, the connected surface further comprises a body fat sensor 782 that measures percent body fat of the user as the user stands on the connected surface. The body fat sensor 782 is integrated into the connect surface such that use of the body fat sensor does not require the user to perform extra steps beyond standing on the connected surface. Percent body fat is an effective statistic in contributing to an overall understanding of overall health, especially when paired with weight and balance measurements.

Optionally, the body fat sensor 782 utilizes the method of near-infrared interactance comprising at least one infrared emitter and at least one photo detector. A beam of infrared light is transmitted into the user's body, often through the foot. The infrared light is reflected by non-fatty tissue and absorbed by fat tissue. The photo detector captures the reflected infrared light. The ratio of the infrared light returned to the photo detector to the infrared light emitted by the infrared emitter is correlated to the percentage body fat of the user using reference values stored by the data processing unit.

Further variations of the body fat sensor 782 include electrical impedance analysis comprising at least two conductors. A small electric current is sent through the user's body and the impedance between the conductors is measured. The impedance is correlated to body fat percentage of the user based on the user's gender, age, weight, and reference values. In general, fat is anhydrous and a poor conductor of electric current. Conversely, non-fatty tissue, water, and electrolytes are good conductors of electric current. Consequently, the percent body fat increases with increased impedance, thus providing a method of measuring percent body fat.

Referring now to FIG. 8, the connected surface further comprises an electrocardiogram sensor 896 that measures the rate and regularity of heartbeats by interpreting the electrical activity of the heart over a period of time. Accordingly, electrodes contact the skin and the electrical activity of the heart is recorded across the thorax of the user. In some cases of the connected surface, electrodes contact the skin at the user's feet and the measurement is taken using two electrodes.

Optionally, two external electrodes removable from the connected surface such that they can be attached to the user's wrists or held in the hand are electrically coupled to the connected surface either by a wired or wireless connection. In this configuration, the electrocardiogram sensor comprises two electrodes attached to the user's wrists or held in the hand. In further implementations of this configuration, the electrocardiogram sensor comprises four electrodes at both feet and both arms to improve measurement quality.

Optionally, in addition to the previously described two external electrodes, a chest strap comprising six electrodes is electrically coupled to the connected surface either by a wired or wireless connection. In this configuration, ten electrodes are in contact with the body at the desired ten points to provide a Twelve-Lead Electrocardiogram, which is most commonly used in clinical practice. Optionally, the two external electrodes are functionally and electrically connected to the chest strap such that both implements are comprised in a singular implement, thus providing only one external implement to the connected surface to complete a Twelve-Lead Electrocardiogram.

Referring to FIG. 9, the connected surface further comprises a pressure point sensor 907 that identifies pressure points created when the user is standing on the connected surface. Accordingly, the pressure point sensor 907 comprises a clear piece of plastic that is oriented to be parallel to the standing surface and is adequately sized for the user to stand on during normal operation of the connected surface. Further, the clear piece of plastic acts as a waveguide for an infrared emitter that emits infrared light into the clear piece of plastic, wherein the infrared light is contained within the internal boundaries of the clear piece of plastic when it is sufficiently flat. Further, the pressure point sensor comprises at least one infrared photo detector positioned on the opposite-side of the clear piece of plastic from the user. Consequently, when the user stands on the pressure point sensor, the clear piece of plastic is deformed in the positions where pressure points exist. The deformation of the clear piece of plastic allows the infrared light to escape the internal boundaries of the plastic, thus becoming visible to the infrared photo detector. The positions where infrared light is detected are indicative of pressure points created by the user as he/she stands on the pressure point sensor and connected surface.

As shown in FIG. 10, the connected surface further comprises at least one temperature sensor 1012 to measure the skin surface temperature of the user during normal use of the connected surface. Optionally, the temperature sensor 1012 comprises multiple embodiments and alternatives that will be described below.

In some embodiments, the temperature sensor is at least one thermocouple, wherein the thermocouple comprises two different conductors, typically metal alloys, that produce a voltage proportional to a temperature difference between either end of the pair of conductors. Optionally, the temperature sensor is at least one thermistor, wherein the thermistor is a resistor that has a certain resistance, which varies significantly with temperature. Thermistors are generally comprised of a ceramic or polymer material.

Optionally, the temperature sensor is at least one resistance temperature detector (RTD), wherein the RTD exploits a predictable change in electrical resistance that is dependent upon a change in temperature. Often, the material of the RTD is platinum. Alternatively, the temperature sensor is at least one infrared temperature sensor, wherein the temperature of an object is determined by a portion of thermal radiation referred to as blackbody radiation emitted by the object, such that knowing the infrared energy emitted and the object's emissivity allows for the determination of the object's temperature.

Optionally, the temperature sensor is at least one thermopile, wherein the thermopile converts thermal energy into electrical energy and is comprised of one or more thermocouples connected in series or parallel. Optionally, the temperature sensor is at least one thermostat, wherein the thermostat comprises two different metals that are bonded together to form a bi-metallic strip, such that the difference in linear expansion rates causes a mechanical bending movement when heat is applied. In some embodiments, the temperature sensor is at least one silicon bandgap temperature sensor, wherein the forward voltage of a silicon diode is dependent on temperature, and the temperature is determined by comparing bandgap voltages at two different currents.

Referring now to FIG. 11, the connected surface is comprised as the base unit 1125 in a user identification system that further comprises a mobile unit 1148 and the human body 1130. The data processing unit of the connected surface 1125 operates a capacitive touch sensor that constantly monitors for a touch input, such as the user stepping on the connected surface. The data processing unit of the connected surface then transmits a signal at a certain frequency to the mobile unit using the human body 1130 as a capacitive coupler. The human body's capacitance allows it to transmit signals at different frequencies simultaneously as a capacitive coupler. Additionally, the mobile unit 1148 comprises a data processing unit. The mobile unit 1148 receives the signal from the connected surface, base unit 1125, indicating the user is in contact with the connected surface and transmits a response signal at a different frequency than the signal sent from the connected surface. The response signal identifies the mobile unit 1148 using a unique identification code, thus identifying the user to the connected surface 1125. Since the frequencies of the two signals differ, the signals can be sent simultaneously allowing for simultaneous identification of the user.

Optionally, the mobile unit 1148 of the user identification system is a toothbrush comprising a data processing unit such that the toothbrush receives the signal from the connected surface through capacitive coupling of the human body. This configuration allows the connected surface to identify the user while the user brushes his/her teeth and associate collected data with the identified user.

Optionally, the mobile unit 1148 is a dedicated system that is used for the sole purpose of identifying the user. Optionally, the mobile unit 1148 is an embedded chip in the user's skin such that the user can consistently be identified by the connected surface. Optionally, the mobile unit 1148 is a tattooed circuit on the user's skin such that the circuit can receive the signal from the connected surface and transmit the identification signal.

In some embodiments, the connected surface further comprises a speaker that is coupled to the data processing unit of the connected surface. Additionally, the data processing unit comprises a voice generator that generates audible speech that is communicated to the user. The voice generator generates speech related to the user's collected data and personal selectable preferences. Optionally, when a user is identified using the user identification system, the voice generator transmits the identified user's related speech through the speaker such that it is audibly transmitted to the user.

Referring now to FIG. 12, the connected surface with sensors including variations described herein is comprised in a system that allows a user to view and monitor the measured data via a data transfer medium 1273, such as a “smartphone”, and/or a network storage device, often known as the “cloud” and hereinafter referred to as the Cloud 1264. Embodiments of the connected surface 1256 comprised in this system include the data transmitter described previously. Accordingly, the system allows the connected surface 1256 to transfer data to the data transfer medium 1273 and/or the Cloud 1264. Additionally, the data transfer medium 1273 may transfer said data to the Cloud 1264 for display and manipulation on further data transfer mediums connected to said Cloud. Alternatively, the Cloud 1264 may transfer said data to the data transfer medium 1273.

In some embodiments, the data transfer medium comprises 1273 a receiver, a transmitter, a data processing unit, and a display. Accordingly, the data processing unit is chosen from the group microprocessor, microcontroller, field programmable gate array (FPGA), digital signal processing unit (DSP), application specific integrated circuit (ASIC), programmable logic, and combinations thereof. The data processing unit comprises a collector, storage medium, and a processor.

Moreover, the storage medium of the data processing unit is comprised of volatile memory and non-volatile memory, wherein volatile memory is used for short-term storage and processing, and non-volatile memory is used for long-term storage. Accordingly, in some embodiments, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Optionally, in some embodiments, non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

Further still, the processor of the data processing unit is chosen from the group microprocessor and microcontroller.

Additionally, the receiver of the data transfer medium is chosen from the group universal serial bus (USB), serial port, wired Ethernet port, radio frequency, microwave communication, infrared short-range communication, near field communication, and Bluetooth. Often, the receiver of the data transfer medium receives at least one signal from the data transmitter of the connected surface.

Optionally, the data transfer medium is chosen from the group personal computer, tablet computer, mobile phone (i.e. “smartphone”), television, dedicated system, charging station, network router, and web-enabled server.

Optionally, the transmitter of the data transfer medium is chosen from the group universal serial bus (USB), serial port, wired Ethernet port, radio frequency, microwave communication, infrared short-range communication, near field communication, and Bluetooth.

Additionally, the display of the data transfer medium converts signals into user-readable formats.

In some embodiments, the Cloud 1264 is connected to a network, wherein the network is chosen from the group Internet or intranet such that an intranet is a network managed and accessed by an internal organization and is not accessible to the outside world. The network is utilized by the Cloud 1264 for receiving and transmitting data. The mode for receiving and transmitting data through the network is chosen from the group universal serial bus (USB), serial port, wired Ethernet port, radio frequency, microwave communication, infrared short-range communication, near field communication, and Bluetooth.

Additionally, the Cloud processes data using at least one microprocessor, at least one microcontroller, or a combination thereof. The storage of data is comprised of volatile memory and non-volatile memory, wherein volatile memory is used for short-term storage and processing, and non-volatile memory is used for long-term storage. Accordingly, volatile memory is chosen from the group random-access memory (RAM), dynamic random-access memory (DRAM), double data rate synchronous dynamic random-access memory (DDR SDRAM), static random-access memory (SRAM), thyristor random-access memory (T-RAM), zero-capacitor random-access memory (Z-RAM), and twin transistor random-access memory (TTRAM). Optionally, non-volatile memory is chosen from the group read-only memory (ROM), programmable read-only memory (PROM), erasable programmable read-only memory (EPROM), electrically erasable programmable read-only memory (EEPROM), flash memory, ferroelectric random-access memory (FeRAM), magnetoresistive random-access memory (MRAM), phase-change memory (PRAM), conductive-bridging random-access memory (CBRAM), silicon-oxide-nitride-oxide-silicon memory (SONOS), resistive random-access memory (RRAM), racetrack memory, nano-random-access memory (NRAM), and Millipede memory.

The Cloud, optionally, is a network server primarily used for storing and processing data. Optionally, the Cloud is comprised of more than one network server such that the network servers operate in conjunction to increase the storing and processing capabilities of the Cloud. Alternatively, the Cloud is provided as a service such that it is physically located at a location separate from the user, and the service provided is the storing and processing of data.

In some embodiments, the system comprising the connected surface facilitates the user's participation in social games related to the data collected by the sensors of the connected surface. Participation in said social games is accomplished passively through the collection of data by the sensors of the connected surface over a period of time, rather than participation by real-time user input. Optionally, the social games consist of goals to be accomplished, competitive games between multiple users or between a singular user and a computer generated user, and challenges to complete specified milestones.

Participation in social games is accomplished through a plurality of different user groups. The first user group for participation is a closed loop user group, which is accomplished on a specific data transfer medium and participation is limited to the users of said specific data transfer medium. The second user group for participation is a networked user group, which is accomplished over a network that connects a plurality of data transfer mediums. Networked user groups are further defined as including users belonging to a certain group defined through social media or other means. The third user group for participation is a global user group, which is a user group that anyone can join and participate in. The global user group, in some embodiments, may be sponsored or promoted by a particular entity as a form of advertisement or incentive to the users of the global user group.

Participation in social games may be incentivized with an offered reward to encourage participation of members of a user group. Rewards may include coupons, discounts on goods or services, virtual currency, insurance discounts, and customized incentives. Rewards have the advantage of being given based off of passive data collected by sensors, thus rewarding users for health compliance and health statistics.

It will be understood that the embodiments described herein are not limited in their application to the details of the teachings and descriptions set forth, or as illustrated in the accompanying figures. Rather, it will be understood that connected surface with sensors, as taught and described according to multiple embodiments disclosed herein, is capable of other embodiments and of being practiced or carried out in various ways.

Also, it is to be understood that the phraseology and terminology used herein is for the purpose of description and should not be regarded as limiting. The use herein of “including,” “comprising,” “i.e.,” “containing,” or “having,” and variations of those words is meant to encompass the items listed thereafter, and equivalents of those, as well as additional items.

Accordingly, the descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

What is claimed is:

1. A connected surface, comprising:
a surface that is configured to allow a user to stand on said surface;
a data processing unit having at least one collector that is configured to collect data, a storage medium that is configured to store data, and at least one processor that is configured to process data;
a transmitter that is configured to transmit data; and
at least one sensor that is configured to measure at least one health statistic.

2. The connected surface of claim 1, wherein the surface is chosen from the group consisting of flooring, a bath mat, a weight scale, a rug, a dedicated system, and any combination thereof.

3. The connected surface of claim 1, wherein the transmitter is chosen from the group consisting of a universal serial bus, serial port, wired Ethernet port, radio frequency, microwave communication, infrared short-range communication, near field communication, Bluetooth, Wi-Fi, and any combination thereof.

4. The connected surface of claim 3, wherein the transmitter is further configured to receive data.

5. The connected surface of claim 1, wherein at least one health statistic is chosen from the group consisting of heart rate, blood oxygen saturation, respiration rate, blood pressure, bone density, weight, percent body fat, skin ulcers, stress level, pressure points, electrical heart activity, thermal imaging, skin surface temperature, and any combination thereof.

6. The connected surface of claim 1, wherein at least one sensor is an oximetry sensor that is configured to measure at least one health statistic chosen from the group consisting of blood oxygen saturation, heart rate, respiration rate, blood pressure, blood flow, and any combination thereof.

7. The connected surface of claim 1, wherein at least one sensor is an ultrasonic sensor that is configured to measure bone density.

8. The connected surface of claim 1, wherein at least one sensor is a weight sensor that is configured to measure at least one health statistic chosen from the group consisting of weight, balance, and any combination thereof.

9. The connected surface of claim 1, wherein at least one sensor is a thermal imaging sensor that is configured to identify skin ulcers.

10. The connected surface of claim 1, wherein at least one sensor is a galvanic skin response sensor that is configured to measure the moisture level of skin of the user.

11. The connected surface of claim 1, wherein at least one sensor is a body fat sensor that is configured to measure percent body fat.

12. The connected surface of claim 1, wherein at least one sensor is an electrocardiogram sensor that is configured to measure electrical heart activity.

13. The connected surface of claim 1, wherein at least one sensor is a pressure point sensor that is configured to identify pressure points created by the user standing on said surface.

14. The connected surface of claim 1, wherein at least one sensor is a temperature sensor that is configured to measure skin surface temperature.

15. The connected surface of claim 1, wherein at least one sensor is chosen from the group consisting of an oximetry sensor, ultrasonic sensor, weight sensor, thermal imaging sensor, galvanic skin response sensor, body fat sensor, electrocardiogram sensor, pressure point sensor, temperature sensor, and any combination thereof.

16. A connected surface system, comprising:
a connected surface that is configured to act as a base unit having a surface that is configured to allow a user to stand on said surface, a data processing unit that is configured to store and process data and detect touch input from the user, a transmitter that is configured to transmit data, and at least one sensor that is configured to measure at least one health statistic; and
a mobile unit having a data processing unit that is configured to store and process data and communicate with the connected surface via capacitive coupling,
wherein both the connected surface and the mobile unit are in contact with the human body of the user such that the human body acts as a capacitive coupler and is arranged to allow for the transmission of at least one signal.

17. The connected surface system of claim 16, wherein the connected surface further comprises at least one speaker that is electrically coupled to the data processing unit of the connected surface such that the speaker and data processing unit, both of the connected surface, operate in conjunction to generate audible speech related to the user.

18. A connected surface system, comprising:
a connected surface having a surface that is configured to allow a user to stand on said surface, a data processing unit that is configured to store and process data and detect touch input from the user, a transmitter that is configured to transmit data, and at least one sensor that is configured to measure at least one health statistic; and
a cloud computing network having at least one data processing unit that is configured to store and process data and a transceiver that is configured to receive and transmit data.

19. The connected surface system of claim 18, further comprising a data transfer medium having a transceiver that is configured to receive and transmit data, a data processing unit that is configured to store and process data, and a display that is configured to show data.

20. The connected surface system of claim 19, wherein the data transfer medium further comprises a user interface that is configured to facilitate participation in social games such that participation is accomplished passively through data collection of the toothbrush over a period of time.
